(19) United States
(12) Patent Application Publication
(10) Pub. No.: US 2015/0269336 A1
(43) Pub. Date: Sep. 24, 2015

(54) method for selecting features of EEG signals based on decision tree

(71) Applicant: Beijing University of Technology, Beijing (CN)

(72) Inventors: Lijuan Duan, Beijing (CN); Hui Ge, Beijing (CN); Zhen Yang, Beijing (CN); Yuanhua Qiao, Beijing (CN); Wei Ma, Beijing (CN); Haiyan Zhou, Beijing (CN)

(21) Appl. No.: 14/583,127

(22) Filed: Dec. 25, 2014

(30) Foreign Application Priority Data
Mar. 24, 2014 (CN) 201410112806.X

(51) Int. Cl. G06F 19/00; G06N 99/00; A61B 5/0484; A61B 5/04; A61B 5/00; G06N 5/04; A61B 5/0478

(52) U.S. Cl. CPC G06F 19/345; G06N 5/04; G06N 99/005; A61B 5/0478; A61B 5/04012; A61B 5/7267; A61B 5/0484

(57) ABSTRACT

The present invention relates to a method for selecting features of EEG signals based on a decision tree: firstly, acquired multi-channel EEG signals are pre-processed, and then the pre-processed EEG signals are performed with feature extraction by utilizing principal component analysis, to obtain a analysis data set matrix with decreased dimensions; superior column vectors are obtained through analyzing from the analysis data set matrix with decreased dimensions by utilizing a decision tree algorithm, and all the superior column vectors are jointed with the number of the columns increased and the number of the rows unchanged, to be reorganized into a final superior feature data matrix; finally, the reorganized superior feature data matrix is input to a support vector machine (SVM) classifier, to perform a classification on the EEG signals, to obtain a classification accuracy. In the present invention, superior features are selected by utilizing a decision tree, to avoid influence of subjective factors during the selection, so that the selection is more objective and with a higher classification accuracy. The average classification accuracy through the present invention may reach 89.1%, increased by 0.9% compared to the conventional superior electrode reorganization.

### CROSS-REFERENCE TO RELATED APPLICATION

This application claims the priority benefit of Chinese patent application No. 201410112806.x, filed Mar. 24, 2014. The entirety of the above-mentioned patent application is hereby incorporated by reference herein and made a part of specification.

### TECHNICAL FIELD

The present invention relates to a method for analyzing electroencephalogram (EEG) signals in EEG studies, and more particularly, to a method for selecting features of EEG signals based on a decision tree.

### BACKGROUND

A human brain is a rather complex system. Electroencephalograph (EEG) signals are electric discharge activities of neuron cells in the brain that are acquired via electrodes and conductive media covered on a scalp, and contain a large amount of information characterizing human physiological and psychological status. EEG study, which is one of the forefront field of scientific research, involves aspects of acquisition, pre-processing, processing, applications of EEG signals and the like, and plays an important role in many fields such as cognitive science, neuroscience, psychology, pathophysiology, information and signal processing, computer science, biotechnology, biomedical engineering, applied mathematics and the like. As brain science becoming increasingly popular, more and more researchers have devoted to the study boom of EEG signals.

Due to the complexity of the structure of the brain, a brain activity may lead to electric discharge in a certain brain region or a plurality of brain regions. In order to acquire EEG signals in active brain regions more accurately and more thoroughly, researchers need to acquire EEG signals at different locations of the brain of a subject. Currently acquisition devices typically adopt multi-channel approaches, such as commonly-used 40-electrode, 64-electrode, 128-electrode, 256-electrode caps and the like. Since acquisition accuracy of EEG signals is at a level of milliseconds, EEG data of a single electrode has high dimensions of properties, and dimensions of EEG data are even higher when the multi-channel data is processed in parallel. In addition, psychological research has found that different stimuli or experimental tasks will activate different brain functional regions, or more specifically from the view of the physical structure of the brain, different stimuli or experimental tasks may cause neuron cells of different structures in the brain to produce discharge activities. Also shown by features of EEG signals, EEG signals that have been acquired contain much redundant information. Accordingly, in a study of EEG signals, electrode screening is an indispensable part, in consideration either of reducing the dimensions of data, and of removing invalid electrode or redundant information.

Conventional electrode selection (spatial feature selection) methods generally involve superior electrode reorganization. That is, superior electrodes (electrodes which increase or do not decrease overall classification accuracy) are selected based on manual statistical processing of classification effects of individual electrodes. This method has two defects: (1) it relies on subjective experience of electrode selection, which tends to make mistakes; (2) the manual operation is complex, time-consuming and labor-intensive.

### SUMMARY

To overcome the above defects of the conventional electrode selection methods, a method for selecting features of EEG signals based on a decision tree is provided by the present invention. An objective of the present invention is to make feature selection more objective by utilizing a decision tree to automatically select superior properties (superior features), so that a classification accuracy of EEG signals may be improved.

The main idea of the method of the present invention is: pre-processing acquired multi-channel EEG signals; extracting features of pre-processed EEG signals by utilizing principal component analysis (PCA), to obtain an analysis data set matrix with decreased dimensions; inputting the analysis data set matrix with decreased dimensions into a decision tree after the feature extraction, to select superior features; reorganizing the selected superior features via the decision tree; inputting the reorganized superior feature data matrix into a support vector machine (SVM) classifier, to classify the EEG signals, to obtain a classification accuracy.

A method for selecting features of EEG signals based on a decision tree, including the following steps:

step 1, providing a sampling electrode on a brain of a subject; triggering m types of stimulating events; sampling at a predetermined time interval for each type of stimulating events; acquiring p EEG signals of the subject as one sample; repeating the sampling for each type of stimulating events to obtain n samples and m*n sampling row vectors in total;

step 2, taking a part of the m*n sampling row vectors acquired by the sampling electrode as a training sample set, and the other part as a test sample set, wherein, each of the training sample set and the test sample set is a matrix with m rows and p columns;

step 3, providing a plurality of sampling electrodes on the brain of the subject; repeating the step 1 and the step 2 until m*n sampling row vectors are acquired for each of the sampling electrodes;

step 4, jointing training sample sets of all the sampling electrodes with a number of columns increased and a number of rows unchanged, to reorganize them into a matrix of training sample sets; similarly jointing test sample sets of all the electrodes with a number of columns increased and a number of rows unchanged, to reorganize them into a matrix of test sample sets;

step 5, jointing the training sample sets and the test sample sets with a number of columns unchanged and a number of rows increased, to reorganized them into an analysis data set matrix;

step 6, decreasing dimensions of the analysis data set matrix by unitizing principal component analysis, to remove inferior column vector data and form an analysis data set matrix with decreased dimensions; and

step 7, analyzing the analysis data set matrix with decreased dimensions by utilizing a decision tree algorithm to obtain superior column vector data; jointing all the superior column vector data with a number of columns increased and a number of rows unchanged to reorganize them into a final superior feature data matrix.

Preferably, the method for selecting features of EEG signals based on a decision tree further includes:

step 8, inputting the superior feature data matrix in to a support vector machine SVM classifier; classifying the EEG signals with respect to the stimulating events, to obtain a classification accuracy.

Preferably, the method for selecting features of EEG signals based on a decision tree further includes:

evenly dividing the rows of the analysis data set matrix in step 5 into 10 parts; taking 1 part of the 10 parts as effect test data, and the remaining 9 parts as effect training data; performing steps 4˜7, to obtain a classification accuracy;

alternatively taking each one part of the 10 parts of data as effect test data, and the remaining 9 parts as effect training data; repeating steps 4˜7, to obtain 10 classification accuracies;

calculating an average of the 10 classification accuracies, to obtain an averaged classification accuracy

Preferably, in the method for selecting features of EEG signals based on a decision tree, the step 7 includes the following steps:

7.1) extracting one column vector from the analysis data set matrix with decreased dimensions; taking each data as one node; then comparing all the nodes; combining nodes with a same value into one node;

7.2) firstly, for each of the nodes that is not combined with another node, marking it with a label representing a type of stimulating events to which the node belongs, according to a property of a row in which the node is located;

7.3) then for each of the nodes that is combined with another node and has a same property with that of a row in which the combined node is located, marking it with a label representing a type of stimulating events to which the node belongs;

7.4) finally, for each of the nodes that is combined with another node and has a different property with that of a row in which the combined node is located, searching for another column vector with a maximum information gain value with the combined node among the remaining column vectors in the analysis data set matrix with decreased dimensions;

7.5) for the said another column vector, repeating steps 7.1)˜7.4), until all the column vectors with labeled nodes are searched out;

wherein all the column vectors searched out are the superior column vector data.

Preferably, in the method for selecting features of EEG signals based on a decision tree, the step 7 includes the following steps:

7.1) extracting one column vector from the analysis data set matrix with decreased dimensions; taking each data as one node; then comparing all the nodes; combining nodes with a same value into one node;

7.2) firstly, for each of the nodes that is not combined with another node, marking it with a label representing a type of stimulating events to which the node belongs, according to a property of a row in which the node is located;

7.3) then for each of the nodes that is combined with another node and has a same property with that of a row in which the combined node is located, marking it with a label representing a type of stimulating events to which the node belongs;

7.4) finally, for each of the nodes that is combined with another node and has a different property with that of a row in which the combined node is located, searching for another column vector with a maximum information gain value with the combined node among the remaining column vectors in the analysis data set matrix with decreased dimensions; and adding the said column vector into a downstream branch of the combined node;

7.5) for the said another column vector, repeating steps 7.1)˜7.4), until all the column vectors are traversed; for a node of a last column vector which does not satisfy a condition to be labeled, assigning a random type of stimulating events and marking it with a label;

7.6) firstly, classifying labels of nodes in the most lower layer, to obtain a label of a majority type; replacing a label of a parent node of the nodes in the most lower layer with the label of a majority type, and deleting the nodes in the most lower layer, to form a simplified node structure; comparing the simplified parent node and the existing parent node with respect to a corresponding actual type of stimulating events; if an accuracy after simplification is higher, keeping the simplified node structure, and proceeding the simplification from downstream to upstream; if the accuracy after simplification is lower, keeping the existing node structure, and ceasing simplification;

wherein all the column vectors that are searched out and are not deleted through simplification are the superior column vector data.

A method for acquiring superior features of EEG signals based on a decision tree, including the following steps:

step 1, providing a sampling electrode on a brain of a subject; triggering m types of stimulating events; sampling at a predetermined time interval for each type of stimulating events; acquiring p EEG signals of the subject as one sample; repeating the sampling for each type of stimulating events to obtain n samples and m*n sampling row vectors in total;

step 2, taking a part of the m*n sampling row vectors acquired by the sampling electrode as a training sample set, and the other part as a test sample set, wherein, each of the training sample set and the test sample set is a matrix with m rows and p columns;

step 3, providing a plurality of sampling electrodes on the brain of the subject; repeating the step 1 and the step 2 until m*n sampling row vectors are acquired for each of the sampling electrodes;

step 4, jointing training sample sets of all the sampling electrodes with a number of columns increased and a number of rows unchanged, to reorganize them into a matrix of training sample sets; similarly jointing test sample sets of all the electrodes with a number of columns increased and a number of rows unchanged, to reorganize them into a matrix of test sample sets;

step 5, jointing the training sample sets and the test sample sets with a number of columns unchanged and a number of rows increased, to reorganized them into an analysis data set matrix;

step 6, decreasing dimensions of the analysis data set matrix by unitizing principal component analysis, to remove inferior column vector data and form an analysis data set matrix with decreased dimensions;

step 7, analyzing the analysis data set matrix with decreased dimensions by utilizing a decision tree algorithm to obtain superior column vector data; jointing all the superior column vector data with a number of columns increased and a number of rows unchanged to reorganize them into a final superior feature data matrix; and

step 8, inputting the superior feature data matrix in to a support vector machine SVM classifier; classifying the EEG signals with respect to the stimulating events.

Preferably, in the method for acquiring superior features of EEG signals based on a decision tree, evenly dividing the rows of the analysis data set matrix in step 4 into 10 parts; taking 1 part of the 10 parts as effect test data, and the remaining 9 parts as effect training data; performing steps 4˜7, to obtain a classification accuracy;

alternatively taking each one part of the 10 parts of data as effect test data, and the remaining 9 parts as effect training data; repeating steps 4˜7, to obtain 10 classification accuracies;

calculating an average of the 10 classification accuracies, to obtain an averaged classification accuracy

Preferably, in the method for acquiring superior features of EEG signals based on a decision tree, the step 7 includes the following steps:

7.1) extracting one column vector from the analysis data set matrix with decreased dimensions; taking each data as one node; then comparing all the nodes; combining nodes with a same value into one node;

7.2) firstly, for each of the nodes that is not combined with another node, marking it with a label representing a type of stimulating events to which the node belongs, according to a property of a row in which the node is located;

7.3) then for each of the nodes that is combined with another node and has a same property with that of a row in which the combined node is located, marking it with a label representing a type of stimulating events to which the node belongs;

7.4) finally, for each of the nodes that is combined with another node and has a different property with that of a row in which the combined node is located, searching for another column vector with a maximum information gain value with the combined node among the remaining column vectors in the analysis data set matrix with decreased dimensions;

7.5) for the said another column vector, repeating steps 7.1)˜7.4), until all the column vectors with labeled nodes are searched out;

wherein all the column vectors searched out are the superior column vector data.

Preferably, in the method for acquiring superior features of EEG signals based on a decision tree, the step 7 includes the following steps:

7.1) extracting one column vector from the analysis data set matrix with decreased dimensions; taking each data as one node; then comparing all the nodes; combining nodes with a same value into one node;

7.2) firstly, for each of the nodes that is not combined with another node, marking it with a label representing a type of stimulating events to which the node belongs, according to a property of a row in which the node is located;

7.3) then for each of the nodes that is combined with another node and has a same property with that of a row in which the combined node is located, marking it with a label representing a type of stimulating events to which the node belongs;

7.4) finally, for each of the nodes that is combined with another node and has a different property with that of a row in which the combined node is located, searching for another column vector with a maximum information gain value with the combined node among the remaining column vectors in the analysis data set matrix with decreased dimensions; and adding the said column vector into a downstream branch of the combined node;

7.5) for the said another column vector, repeating steps 7.1)˜7.4), until all the column vectors are traversed; for a node of a last column vector which does not satisfy a condition to be labeled, assigning a random type of stimulating events and marking it with a label;

7.6) firstly, classifying labels of nodes in the most lower layer, to obtain a label of a majority type; replacing a label of a parent node of the nodes in the most lower layer with the label of a majority type, and deleting the nodes in the most lower layer, to form a simplified node structure; comparing the simplified parent node and the existing parent node with respect to a corresponding actual type of stimulating events; if an accuracy after simplification is higher, keeping the simplified node structure, and proceeding the simplification from downstream to upstream; if the accuracy after simplification is lower, keeping the existing node structure, and ceasing simplification;

wherein all the column vectors that are searched out and are not deleted through simplification are the superior column vector data.

The invention has the following advantageous effects:

1. in the present invention, superior features are selected by utilizing a decision tree, so that the operation is simple, without human intervention, saving time and labor;

2. in the present invention, superior features are selected by utilizing a decision tree, to avoid influence of subjective factors during the selection, so that the selection is more objective and with a higher classification accuracy. Experiments show that the average classification accuracy of EEG signals through the method of the present invention may reach 89.1%, increased by 0.9% compared to the conventional superior electrode reorganization.

### BRIEF DESCRIPTION OF THE DRAWINGS

FIG. 1 is a flow chart showing a method for selecting features of EEG signals based on a decision tree;

FIG. 2 is a schematic view showing distribution of locations of the sampling electrodes on a scalp surface; and

FIG. 3 is a schematic view showing a structure of sub-trees of the decision tree.

### DETAILED DESCRIPTION

Hereinafter, the present invention is further described in detail in conjunction with accompany drawings and specific embodiments.

FIG. 1 shows a flow chart of the present invention, including the following steps.

Step 1, data acquisition.

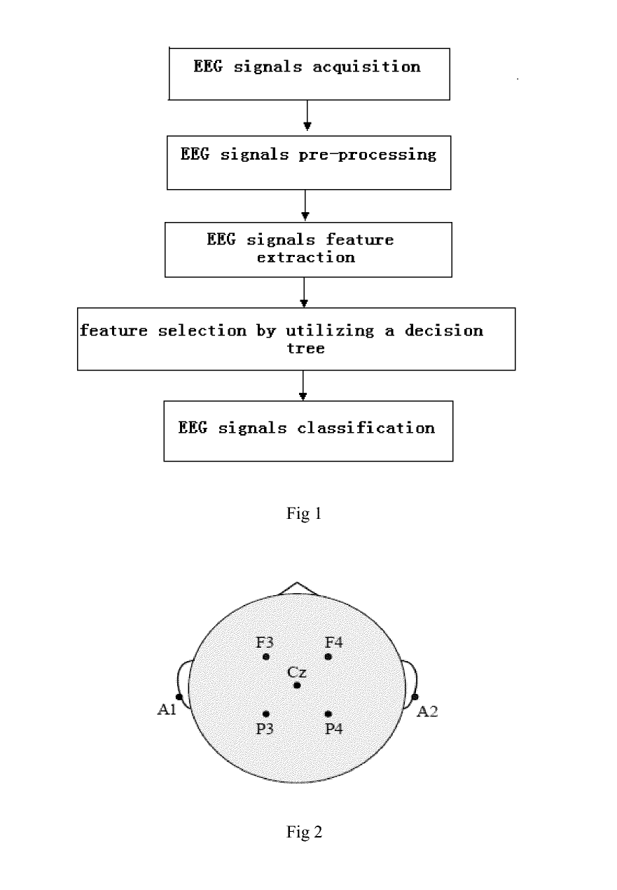

The method of the present invention is applied to BCI 2003 competition standard Data Set Ia. The data is acquired from one healthy subject. An experimental task of the subject is to move up or down a cursor on a screen through imagination. A component induced by the imagination is a slow cortical potential (SCP) at a low frequency, and the cortical potential is recorded by an electrode Cz. Each experiment lasts for 6 seconds. Within 0.5 s˜6 s, a highlighted indication bar is presented above or below the computer screen, prompting the subject to move up or down the cursor in the middle of the screen. A rule for the movement is: when the SCP is positive, the cursor is moved down, and when the SCP is negative, the cursor is moved up. Within 2 s˜5.5 s, the subject receives feedback information on SCP amplitude from the electrode Cz, which is displayed as a bright bar below the screen which has a length in proportion to the SCP amplitude. At this time, EEG signals of the subject are recorded at a sampling rate of 256 Hz, and recording electrodes are respectively located at six positions A1, A2, P3, P4, F3 and F4. Data for final signal processing are two types of EEG signals recorded within 3.5 s in the information feedback phase. Locations of the electrodes are shown in FIG. 2.

Two sets of experiment data are acquired during the experiment: a training data set and a test data set. The training data set is used to training a classifier, and the test data set is used to determining performance of the classifier. Since in the present experiment, only two types of EEG signals are acquired, prediction of classification of the data involves only one or two categories, and the category labels are 0 and 1, where 0 represents a category of signals corresponding to moving down of the cursor, and 1 represents a category of signals corresponding to moving up of the cursor.

Step 2, data preprocessing.

Step 2.1, data reorganization.

An acquired training sample set of one electrode is represented as a b*d matrix, where b represents a sampling size of the training sample set, and d=r*t represents dimensions of the sample set, where r represents a sampling rate, and t represents a sampling time. Acquired training sample sets of all the electrodes are reorganized as one training sample set with the sampling size unchanged and the dimensions increased, that is, reorganized as a b*k matrix, k=a*d, where a represents a number of the electrodes.

An acquired test sample set of one electrode is represented as a e*d matrix, where e represents a sampling size of the test sample set. Test sample sets of all the electrodes are reorganized as an e*k matrix in a similar way to the training sample sets.

Step 2.2, data grouping.

The test data set and the training data set obtained in step 2.1 are jointed as one data set with the dimensions unchanged and the sampling size increased, that is, jointed as an h*k matrix, h=b+d. Then this data set is evenly divided into 10 parts by the sample size, where the training data is a w*k matrix, w=0.9 h, the test data is a g*k matrix, g=0.1 h.

Step 3, feature extraction.

Feature extraction is respectively performed to the training data and the test data by utilizing PCA, to obtain an analysis data set matrix with decreased dimensions. That is, each of the two data sets is decreased from k dimensions to i dimensions, or data of each electrode is decreased from d dimensions to q dimensions, where i=a*q.

Step 4, feature selection.

The analysis data set matrix with decreased dimensions after the feature extraction is input to a decision tree, to select superior features. The process includes the following two steps:

Step 4.1, decision tree construction.

A tree is created from a single node representing a training sample. A property is stored at a node. If the samples all belong to a same category, the node is a leaf and marked as a category. Otherwise, a property is selected by which the samples may be best classified, i.e. a property with the highest information gain, by utilizing information gains as heuristic information measured based on entropy. The property becomes a “test” or “determination” property of the node. For each known value of the test property, a branch is created, and the samples are divided accordingly. The above process is repeated, to recursively form a sample decision tree for each division, until one of the following conditions are established:

a first condition: samples of a given node belong to a same category;

a second condition: no property left that may be used to further divide the samples, and in this case, by majority voting, the given node is transformed to a leaf, and marked with a category to which the majority of its samples belong; and

a third condition: there is no sample in a branch created for a known value of a test property, and in this case, a leaf is created for a category to which the majority of its samples belong.

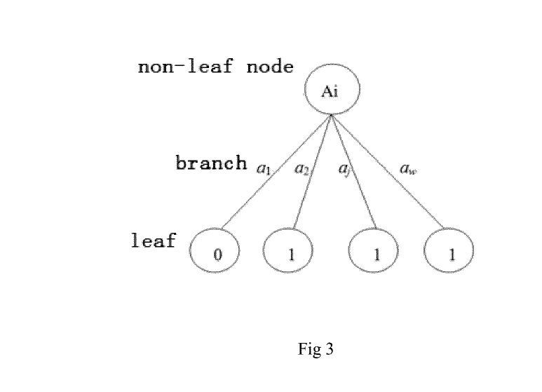

A complete decision tree is constructed which has non-leaf nodes, branches and leaves (leaf nodes) according to the above method. For i-dimension training data, there are i properties accordingly. A property Ar is stored at a node of the tree, where r=1, 2, . . . , d. The samples are divided by Ar=aj, where aj is a known value of the property Ar, j=1, 2, . . . , w. Category labels of samples of the nodes are stored at leafs. A structure of a sub-tree of the decision tree is shown in FIG. 3.

Step 4.2, decision tree trimming.

Each sub-tree of a non-leaf node of a complete decision tree is intended to be replaced with a leaf node, and a category of the leaf node is replaced with a category to which the majority of the training samples covered by the sub-tree belong, thereby a simplified decision tree is generated. Then, on the test data set, performances of the complete decision tree and the simplified decision tree are compared, and if the simplified decision tree makes fewer mistakes on the test data set and the sub-tree does not include another sub-tree having a similar characteristic of reducing misjudgment rate on the test data set after the sub-tree being replaced with a leaf node, the sub-tree may be replaced with a leaf node. All the sub-trees are traversed from top to bottom in this way, until there no sub-tree that may be replaced to further improve the performance on the test data set, and the trimming is terminated, to obtain a final simplified tree.

At this time, the simplified tree has c non-leaf nodes, corresponding to c properties (i.e. features) in the data set, and the c features are referred to as superior features (features contributing more to classification effect), that is, superior column vector data. All the superior column vectors are jointed with the number of the columns increased and the number of the rows unchanged, to be reorganized into a final superior feature data matrix.

Step 5, EEG signal classification.

The superior feature data matrix is input to a support vector machine (SVM) classifier, to perform a classification on the EEG signals with respect to stimulating events, to obtain a classification accuracy.

Step 6, classification accuracy calculating.

Each one part of the 10 parts of data in step 2.2 alternatively taken as test data, and the remaining 9 parts of the data as training data, steps 3 to 5 are repeated, to perform 10 times of experiments, and an average of the classification accuracies of the 10 times of experiments is obtained, to obtain a final classification accuracy.

In order to verify the effectiveness of the present invention, and compare the performance of the present invention with that of the conventional classification method by utilizing superior electrode reorganization, a set of comparative experiments are conducted, and the accuracies of the two classification methods are shown in Table 1.

TABLE 1
comparison between accuracies of the present invention and those of the
conventional classification method

| sequence | classification accuracy of | classification accuracy of the |
|---|---|---|
| number of | the method of | conventional |
| experiment | the present invention (%) | classification method (%) |
| 1 | 91.0714 | 87.5 |
| 2 | 89.2857 | 85.7143 |
| 3 | 89.2857 | 85.7143 |
| 4 | 92.8571 | 89.2857 |
| 5 | 91.0714 | 85.7143 |
| 6 | 76.7857 | 80.3571 |
| 7 | 89.4737 | 91.2281 |
| 8 | 91.0714 | 96.4286 |
| 9 | 89.2857 | 91.4286 |
| 10 | 91.0714 | 89.2857 |
| average | 89.1259 | 88.2300 |

It can be seen from Table 1 that, the classification accuracies of EEG signals through the method of the present invention are all above 90%, except the 6^{th} experiment, and the average accuracy is 89.1%; and the average classification accuracy of EEG signals through the method by utilizing conventional superior electrode reorganization is 88.2%. Thus, the classification accuracy is increased by 0.9% through the method of the present invention, compared with the conventional method.

Although the embodiments of the present invention have been disclosed as above, they are not limited merely to those set forth in the description and the embodiments, and they may be applied to various fields suitable for the present invention. For those skilled in the art, other modifications may be easily achieved, and the present invention is not limited to the particular details and illustrations shown and described herein, without departing from the general concept defined by the claims and their equivalents.

What is claimed is:

1. A method for selecting features of EEG signals based on a decision tree, characterized in that, the method comprises the following steps:
step 1, providing a sampling electrode on a brain of a subject; triggering m types of stimulating events; sampling at a predetermined time interval for each type of stimulating events; acquiring p EEG signals of the subject as one sample; repeating the sampling for each type of stimulating events to obtain n samples and m*n sampling row vectors in total;
step 2, taking a part of the m*n sampling row vectors acquired by the sampling electrode as a training sample set, and the other part as a test sample set, wherein, each of the training sample set and the test sample set is a matrix with m rows and p columns;
step 3, providing a plurality of sampling electrodes on the brain of the subject; repeating the step 1 and the step 2 until m*n sampling row vectors are acquired for each of the sampling electrodes;
step 4, jointing training sample sets of all the sampling electrodes with a number of columns increased and a number of rows unchanged, to reorganize them into a matrix of training sample sets; similarly jointing test sample sets of all the electrodes with a number of columns increased and a number of rows unchanged, to reorganize them into a matrix of test sample sets;
step 5, jointing the training sample sets and the test sample sets with a number of columns unchanged and a number of rows increased, to reorganized them into an analysis data set matrix;
step 6, decreasing dimensions of the analysis data set matrix by unitizing principal component analysis, to remove inferior column vector data and form an analysis data set matrix with decreased dimensions; and
step 7, analyzing the analysis data set matrix with decreased dimensions by utilizing a decision tree algorithm to obtain superior column vector data; jointing all the superior column vector data with a number of columns increased and a number of rows unchanged to reorganize them into a final superior feature data matrix.

2. The method for selecting features of EEG signals based on a decision tree as recited in claim 1, characterized in that, the method further comprises:
step 8, inputting the superior feature data matrix in to a support vector machine SVM classifier; classifying the EEG signals with respect to the stimulating events, to obtain a classification accuracy.

3. The method for selecting features of EEG signals based on a decision tree as recited in claim 2, characterized in that, the method further comprises:
evenly dividing the rows of the analysis data set matrix in step 5 into 10 parts; taking 1 part of the 10 parts as effect test data, and the remaining 9 parts as effect training data; performing steps 4˜7, to obtain a classification accuracy;
alternatively taking each one part of the 10 parts of data as effect test data, and the remaining 9 parts as effect training data; repeating steps 4˜7, to obtain 10 classification accuracies;
calculating an average of the 10 classification accuracies, to obtain an averaged classification accuracy

4. The method for selecting features of EEG signals based on a decision tree as recited in claim 1, characterized in that, the step 7 comprises the following steps:
7.1) extracting one column vector from the analysis data set matrix with decreased dimensions; taking each data as one node; then comparing all the nodes; combining nodes with a same value into one node;
7.2) firstly, for each of the nodes that is not combined with another node, marking it with a label representing a type of stimulating events to which the node belongs, according to a property of a row in which the node is located;
7.3) then for each of the nodes that is combined with another node and has a same property with that of a row in which the combined node is located, marking it with a label representing a type of stimulating events to which the node belongs;
7.4) finally, for each of the nodes that is combined with another node and has a different property with that of a row in which the combined node is located, searching for another column vector with a maximum information gain value with the combined node among the remaining column vectors in the analysis data set matrix with decreased dimensions;
7.5) for the said another column vector, repeating steps 7.1)˜7.4), until all the column vectors with labeled nodes are searched out;
wherein all the column vectors searched out are the superior column vector data.

5. The method for selecting features of EEG signals based on a decision tree as recited in claim 1, characterized in that, the step 7 comprises the following steps:
7.1) extracting one column vector from the analysis data set matrix with decreased dimensions; taking each data as one node; then comparing all the nodes; combining nodes with a same value into one node;
7.2) firstly, for each of the nodes that is not combined with another node, marking it with a label representing a type of stimulating events to which the node belongs, according to a property of a row in which the node is located;
7.3) then for each of the nodes that is combined with another node and has a same property with that of a row in which the combined node is located, marking it with a label representing a type of stimulating events to which the node belongs;
7.4) finally, for each of the nodes that is combined with another node and has a different property with that of a row in which the combined node is located, searching for another column vector with a maximum information gain value with the combined node among the remaining column vectors in the analysis data set matrix with decreased dimensions; and adding the said column vector into a downstream branch of the combined node;
7.5) for the said another column vector, repeating steps 7.1)˜7.4), until all the column vectors are traversed; for a node of a last column vector which does not satisfy a condition to be labeled, assigning a random type of stimulating events and marking it with a label;
7.6) firstly, classifying labels of nodes in the most lower layer, to obtain a label of a majority type; replacing a label of a parent node of the nodes in the most lower layer with the label of a majority type, and deleting the nodes in the most lower layer, to form a simplified node structure; comparing the simplified parent node and the existing parent node with respect to a corresponding actual type of stimulating events; if an accuracy after simplification is higher, keeping the simplified node structure, and proceeding the simplification from downstream to upstream; if the accuracy after simplification is lower, keeping the existing node structure, and ceasing simplification;
wherein all the column vectors that are searched out and are not deleted through simplification are the superior column vector data.

6. A method for acquiring superior features of EEG signals based on a decision tree, characterized in that, the method comprises the following steps:
step 1, providing a sampling electrode on a brain of a subject; triggering m types of stimulating events; sampling at a predetermined time interval for each type of stimulating events; acquiring p EEG signals of the subject as one sample; repeating the sampling for each type of stimulating events to obtain n samples and m*n sampling row vectors in total;
step 2, taking a part of the m*n sampling row vectors acquired by the sampling electrode as a training sample set, and the other part as a test sample set, wherein, each of the training sample set and the test sample set is a matrix with m rows and p columns;
step 3, providing a plurality of sampling electrodes on the brain of the subject; repeating the step 1 and the step 2 until m*n sampling row vectors are acquired for each of the sampling electrodes;
step 4, jointing training sample sets of all the sampling electrodes with a number of columns increased and a number of rows unchanged, to reorganize them into a matrix of training sample sets; similarly jointing test sample sets of all the electrodes with a number of columns increased and a number of rows unchanged, to reorganize them into a matrix of test sample sets;
step 5, jointing the training sample sets and the test sample sets with a number of columns unchanged and a number of rows increased, to reorganized them into an analysis data set matrix;
step 6, decreasing dimensions of the analysis data set matrix by unitizing principal component analysis, to remove inferior column vector data and form an analysis data set matrix with decreased dimensions;
step 7, analyzing the analysis data set matrix with decreased dimensions by utilizing a decision tree algorithm to obtain superior column vector data; jointing all the superior column vector data with a number of columns increased and a number of rows unchanged to reorganize them into a final superior feature data matrix; and
step 8, inputting the superior feature data matrix in to a support vector machine SVM classifier; classifying the EEG signals with respect to the stimulating events.

7. The method for acquiring superior features of EEG signals based on a decision tree as recited in claim 2, characterized in that,
evenly dividing the rows of the analysis data set matrix in step 4 into 10 parts; taking 1 part of the 10 parts as effect test data, and the remaining 9 parts as effect training data; performing steps 4˜7, to obtain a classification accuracy;
alternatively taking each one part of the 10 parts of data as effect test data, and the remaining 9 parts as effect training data; repeating steps 4˜7, to obtain 10 classification accuracies;
calculating an average of the 10 classification accuracies, to obtain an averaged classification accuracy

8. The method for acquiring superior features of EEG signals based on a decision tree as recited in claim 1, characterized in that, the step 7 comprises the following steps:
7.1) extracting one column vector from the analysis data set matrix with decreased dimensions; taking each data as one node; then comparing all the nodes; combining nodes with a same value into one node;
7.2) firstly, for each of the nodes that is not combined with another node, marking it with a label representing a type of stimulating events to which the node belongs, according to a property of a row in which the node is located;
7.3) then for each of the nodes that is combined with another node and has a same property with that of a row in which the combined node is located, marking it with a label representing a type of stimulating events to which the node belongs;
7.4) finally, for each of the nodes that is combined with another node and has a different property with that of a row in which the combined node is located, searching for another column vector with a maximum information gain value with the combined node among the remaining column vectors in the analysis data set matrix with decreased dimensions;
7.5) for the said another column vector, repeating steps 7.1)˜7.4), until all the column vectors with labeled nodes are searched out;
wherein all the column vectors searched out are the superior column vector data.

9. The method for acquiring superior features of EEG signals based on a decision tree as recited in claim 1, characterized in that, the step 7 comprises the following steps:
7.1) extracting one column vector from the analysis data set matrix with decreased dimensions; taking each data as one node; then comparing all the nodes; combining nodes with a same value into one node;
7.2) firstly, for each of the nodes that is not combined with another node, marking it with a label representing a type of stimulating events to which the node belongs, according to a property of a row in which the node is located;
7.3) then for each of the nodes that is combined with another node and has a same property with that of a row in which the combined node is located, marking it with a label representing a type of stimulating events to which the node belongs;
7.4) finally, for each of the nodes that is combined with another node and has a different property with that of a row in which the combined node is located, searching for another column vector with a maximum information gain value with the combined node among the remaining column vectors in the analysis data set matrix with decreased dimensions; and adding the said column vector into a downstream branch of the combined node;
7.5) for the said another column vector, repeating steps 7.1)˜7.4), until all the column vectors are traversed; for a node of a last column vector which does not satisfy a condition to be labeled, assigning a random type of stimulating events and marking it with a label;
7.6) firstly, classifying labels of nodes in the most lower layer, to obtain a label of a majority type; replacing a label of a parent node of the nodes in the most lower layer with the label of a majority type, and deleting the nodes in the most lower layer, to form a simplified node structure; comparing the simplified parent node and the existing parent node with respect to a corresponding actual type of stimulating events; if an accuracy after simplification is higher, keeping the simplified node structure, and proceeding the simplification from downstream to upstream; if the accuracy after simplification is lower, keeping the existing node structure, and ceasing simplification;
wherein all the column vectors that are searched out and are not deleted through simplification are the superior column vector data.
